(19) United States
(12) Patent Application Publication
(10) Pub. No.: US 2025/0367018 A1
(43) Pub. Date: Dec. 4, 2025

(54) Penile Traction Device

(71) Applicant: Sergio Castaneda, Las Vegas, NV (US)

(72) Inventor: Sergio Castaneda, Las Vegas, NV (US)

(21) Appl. No.: 18/827,753

(22) Filed: Sep. 8, 2024

Related U.S. Application Data
(60) Provisional application No. 63/652,666, filed on May 28, 2024.

(51) Int. Cl. A61F 5/41

(52) U.S. Cl. CPC A61F 5/41; A61F 2005/412

(57) ABSTRACT

An improved penile traction device includes at least one generally U-shaped yoke, each U-shaped yoke having a vertex and two arms; at least one flexible and bendable penile ring capable of interfacing with a human penis; and at least one twist lock for constricting the at least one flexible and bendable penile ring. The twist lock includes a base unit having a plurality of cam bodies and a twistable ring which engages the plurality of cam bodies to constrict at least one bendable and flexible penile ring. The improved penile traction device can be used with multiple distraction treatment methods. The invention is also capable of being utilized within specialized vacuum cylinder devices, thus providing two operational attributes in one device—both a vacuum device and a traction device.

### CROSS-REFERENCE TO RELATED APPLICATIONS

The present utility patent application claims priority benefit of the U.S. provisional application Ser. No. 63/652,666 titled “Traction Device” filed on May 28, 2024 under 35 U.S.C. § 119 (e). The contents of this related nonprovisional application are incorporated herein by reference for all purposes to the extent that such subject matter is not inconsistent herewith or limiting hereof.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### BACKGROUND OF THE INVENTION

### 1. Field of the Invention

One or more embodiments of the invention generally relate to penile traction devices. More particularly, embodiments of the invention relate to an improved penile traction and enhancement device capable of being implemented in a greater number of applications.

### 2. Description of the Related Art

Male erectile dysfunction is a common medical problem. It has many roots both physiological and psychological, but its effects may be devastating for both the individual and the individual's partner. Erectile dysfunction and/or penile shortening may be the result of common aging to surgical procedures such as radical prostatectomy and transurethral resection of the prostate.

Peyronie's disease (PD) is a connective tissue disorder involving the tunica albuginea layer of the penis. The fibrotic process that occurs may result in the formation of plaque, one of the classic findings on physical examination of the patient. Men afflicted by this disorder may present with pain, deformity (such as penile curvature or penile shortening) and/or erectile dysfunction (ED).

Medical devices are an attractive alternative to surgery due to their potential to avoid invasive procedures. Moreover, medical devices work to treat multiple aspects of the disease, and to intervene before the effects of Peyronie's disease are no longer reversible.

Several methods of treatment for erectile dysfunction and Peyronie's disease are now in use ranging from drug therapy to the use of medical devices. In addition to erectile dysfunction treatment, many males wish to enhance their penis size and sexual performance, to which analogous treatments are available.

Among the more extreme methods of erectile dysfunction treatment is the use of relatively stiff flexible rods surgically implanted in the penis. Another extreme method is the implantation of slim tubular balloons in the penis rather than rods which may be inflated with fluid from a reservoir when an erection is desired. Though proven effective, these methods of treatment come with serious risks which deter numerous men from seeking such treatment; and while surgical therapy has proven itself to be a definitive treatment for penile curvature associated with Peyronie's disease, less invasive options are currently a desired alternative.

Penile traction devices are a class of medical device for treating erectile dysfunction and for increasing penile length. These devices usually consist of a plastic support ring, a silicone band, and two dynamic rods. Penile traction therapy works by holding the penis in a cradle and subjecting it to gentle and progressive traction forces that can be achieved by the addition of small metal extensions to the dynamic rods and cradle frame every few weeks. As the penile tissues are slowly yet surely stretched, which causes microscopic tears which, in theory, the body heals by producing cells to repair the gap. With each repair phase, the penile tissues expand. The desired end result being that the penis lengthens and widens.

Penile traction devices have limitations though. Most notably, penile traction devices can be highly uncomfortable and painful to use. Presently, there exists a need for an improved penile traction device which is capable of providing the results of existing traction devices, but is more comfortable to use.

### SUMMARY

The present invention introduces a new technology, which fulfils the need for a more versatile penile traction device which provides greater comfort and reduced anxiety for a user. At its essence, the improved penile traction device comprises a yoke assembly, a flexible ring, and a means for constricting the flexible ring. The improved penile traction device and method is versatile and readily implementable to treat a wide variety of erectile dysfunction problems and may serve to enhance penis length and girth.

It is an object of the invention to provide a versatile penile traction device. In embodiments of the present invention, the improved penile traction device can be used with multiple distraction treatment methods. The invention is also capable of being utilized within specialized vacuum cylinder devices, thus providing two operational attributes in one device—both a vacuum device and a traction device.

Use of the improved penile traction device involves the user preparing and inserting a penis into a flexible and bendable ring, inserting the penis and ring into the twist lock assembly, securing the penis in the yoke assembly by twisting the base unit to apply pressure on the penis, and applying a distractive force to the inserted and secured penis. Additional steps include, but are not limited to, applying a vacuum to the inserted and secured penis and allowing distractive force and vacuum to operate for a therapeutic amount of time.

### BRIEF DESCRIPTION OF THE DRAWINGS

The invention directed by way of example, and not by way of limitation, in the figures of the accompanying drawings and in which like reference numerals refer to similar elements and in which:

FIG. 1 is an exploded perspective view of the exemplary improved penile traction device in accordance with an embodiment of the invention.

FIG. 2 is a perspective view of the exemplary improved penile traction device in accordance with an embodiment of the invention.

FIG. 3A is a perspective view of the exemplary improved penile traction device in accordance with an embodiment of the invention.

FIG. 3B is a perspective view of the exemplary improved penile traction device in accordance with an alternative embodiment of the invention.

FIG. 4A is a side view of the frame unit of the exemplary improved penile traction device in accordance with an embodiment of the invention.

FIG. 4B is a side view of the frame unit of the exemplary improved penile traction device in accordance with an alternative embodiment of the invention.

FIG. 4C is a side view of the frame unit of the exemplary improved penile traction device in accordance with an alternative embodiment of the invention.

FIG. 4D is a side view of the frame unit of the exemplary improved penile traction device in accordance with an alternative embodiment of the invention.

FIG. 5A is a bottom view of the base unit of the exemplary improved penile traction device in accordance with an embodiment of the invention.

FIG. 5B is a bottom view of the base unit of the exemplary improved penile traction device in accordance with an alternative embodiment of the invention.

FIG. 5C is a bottom view of the base unit of the exemplary improved penile traction device in accordance with an alternative embodiment of the invention.

FIG. 6A is a side view of an exemplary improved penile traction device utilized in a penile traction device in accordance with an alternative embodiment of the invention.

FIG. 6B is a side view of an exemplary improved penile traction device utilized in a penile traction device in accordance with an alternative embodiment of the invention.

Unless otherwise indicated illustrations in the figures are not necessarily drawn to scale.

### DETAILED DESCRIPTION OF THE PREFERRED EMBODIMENTS

Terminology used herein is used for the purpose of describing particular embodiments only, and is not intended to limit the scope of the present invention. It must be understood that as used herein and in the appended claims, the singular forms “a,” “an,” and “the” include the plural reference unless the context clearly dictates otherwise. For example, a reference to “an element” is a reference to one or more elements and includes all equivalents known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Language that may be construed to express approximation should be so understood unless the context clearly dictates otherwise.

Unless defined otherwise, all technical and scientific terms used herein have the same meanings as commonly understood by a person of ordinary skill in the art to which this invention belongs. Preferred methods, techniques, devices, and materials are described. But any methods, techniques, devices, or materials similar or equivalent to those described herein may be used in the practice or testing of the present invention. Structures described herein should also be understood to refer to functional equivalents of such structures.

References to “one embodiment,” “an embodiment,” “various embodiments,” etc., may indicate that the embodiment(s) of the invention so described may include particular features, structures, or characteristics. However, not every embodiment necessarily includes the particular features, structures, or characteristics. Further, repeated use of the phrase “in one embodiment,” or “in an exemplary embodiment,” do not necessarily refer to the same embodiment although they may. A description of an embodiment with several components in communication with each other does not imply that all such components are required. On the contrary, a variety of optional components are described to illustrate the wide variety of possible embodiments of the present invention.

As is well known to those skilled in the art, many careful considerations and compromises typically must be made when designing for the optimal manufacture of a commercial implementation of such an improved penile traction device. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The exemplary improved penile traction device will now be described in detail with reference to embodiments thereof as illustrated in the accompanying drawings.

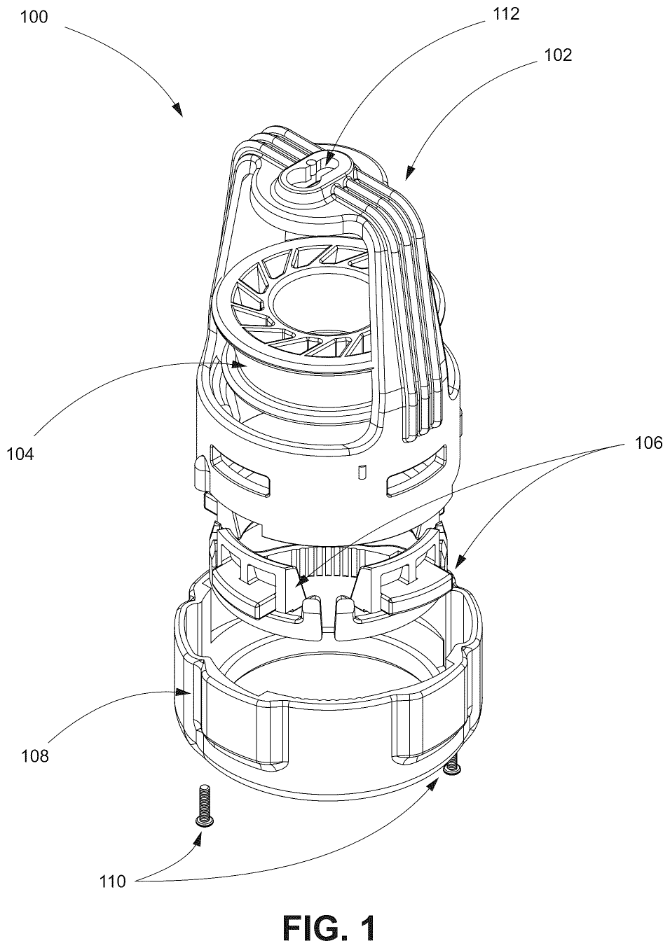

FIG. 1 is an exploded perspective view of the exemplary improved penile traction device 100 in accordance with an embodiment of the invention. In embodiments of the invention, the exemplary improved penile traction device 100 comprises a generally U-shaped yoke assembly 102 having a vertex and two or more arms extending from the vertex, a flexible and bendable penile ring 104, and at least one twistable ring 108 for constricting the at least one flexible and bendable penile ring. In embodiments of the invention, the twist lock for constricting the flexible ring includes a base unit having a plurality of cam bodies 106 and a twistable ring 108 which, when twisted, engages the plurality of cam bodies on the base unit 106 with the flexible and bendable penile ring 104. When assembled, the base unit having a plurality of cam bodies 106 and the twistable ring 108 form a twist lock for constricting the at least one flexible and bendable penile ring 104. The base unit having a plurality of cam bodies 106 and a twistable ring 108 fit together via a series of tabs, grooves, and threads which enable the twistable ring to twist and engage the base unit having a plurality of cam bodies 106.

In embodiments of the invention, the yoke assembly 102 may be made from a medical-grade polycarbonate resin plastic. However, persons skilled in the art will readily appreciate that alternative embodiments may be made from a number of materials such as, but not limited to, metal and metal alloys, clear acrylic plastics, ABS plastic, polycarbonates, polyvinyl chloride (PVC), or various other plastic equivalents. A means for fastening the base unit 108 to the yoke assembly 102 can include two screws 110. Persons skilled in the art will appreciate that other means of fastening the twistable ring 108 to the generally U-shaped yoke assembly 102 can be used.

In embodiments of the invention, the twistable ring 108 may also be made from a medical-grade polycarbonate resin plastic. However, persons skilled in the art will readily appreciate that alternative embodiments may be made from a number of materials such as, but not limited to, metal and metal alloys, clear acrylic plastics, ABS plastic, polycarbonates, polyvinyl chloride (PVC), or various other plastic equivalents. Likewise, the cam bodies 106 may also be made from a medical-grade polycarbonate resin plastic or materials such as, but not limited to, metal and metal alloys, clear acrylic plastics, ABS plastic, polycarbonates, polyvinyl chloride (PVC), or various other plastic equivalents.

An attachment point 112 is located generally at the vertex of the generally U-shaped yoke assembly 102 having a vertex and two or more arms extending from the vertex. The attachment point 112 is capable of interfacing with a penile distraction device. Such an attachment point 112 enables the improved penile traction device 100 to attach to various distraction therapy devices. In one embodiment of the invention, the attachment point 112 is a simple threaded hole for a screw to attach. In other embodiments, the attachment point 112 can be a latching mechanism such as, but not limited to, a carabiner-style clasp. In other embodiments, the attachment point 112 can be a magnetic mechanism. In further embodiments, the attachment point 112 can be a loop through which a cord or cable may attach by a simple knot. Persons skilled in the art will appreciate the numerous possibilities of creating such an attachment point.

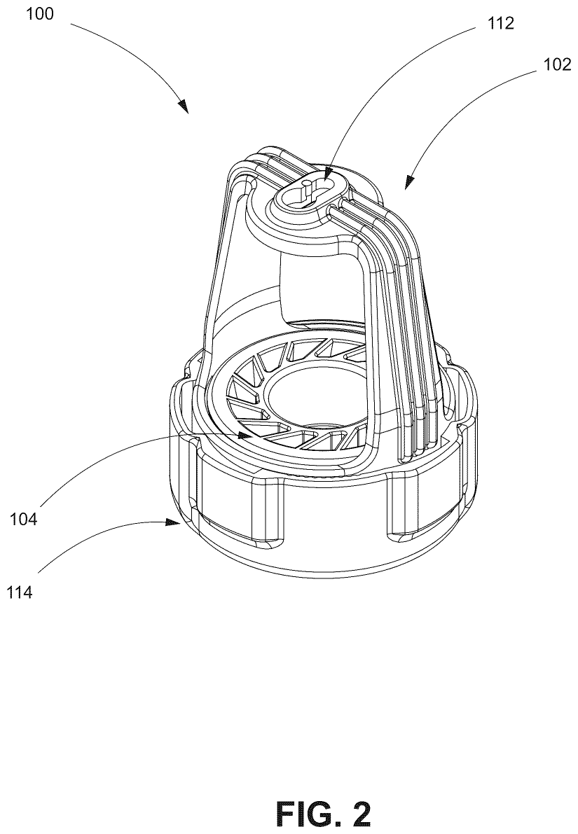

FIG. 2 is a perspective view of the exemplary improved penile traction device 100 in accordance with an embodiment of the invention. In embodiments of the invention, the exemplary improved penile traction device 100 comprises a generally U-shaped yoke assembly 102 having a vertex and two or more arms extending from the vertex, a flexible and bendable penile ring 104, and at least one twist lock 114 for constricting the at least one flexible and bendable penile ring. When assembled, the base unit having a plurality of cam bodies 106 and the twistable ring 108 form a twist lock 114 for constricting the at least one flexible and bendable penile ring 104. The base unit having a plurality of cam bodies 106 and a twistable ring 108 fit together via a series of tabs, grooves, and threads which enable the twistable ring to twist and engage the base unit having a plurality of cam bodies 106. In embodiments of the invention, the at least one twist lock 114 for constricting the at least one flexible and bendable penile ring is adjustable so as to provide sufficient gripping force on a penis so as to remain engaged with a human penis when a distractive force is applied. The twist lock may be held in place by frictional force, or it can be held in place by a simple ratcheting mechanism. The improved penile traction device is designed to be compact, comfortable, and configurable to accommodate penises of varying lengths and girths.

In various embodiments of the invention, the flexible and bendable penile ring 104 is designed in such a way that a user may insert the glans of the penis through a center aperture. The user then can secure the flexible and bendable penile ring to the twist lock 114 which is secured to the generally U-shaped yoke assembly 102. In such embodiments, the improved penile traction device 100 is capable of remaining secured to a human penis while distractive forces are applied. By way of example, and not limitation, a weighted device can be attached to the yoke assembly whereas the weighted device can simply hang from an improved traction device attached to a human penis so as to apply gravitational distraction force.

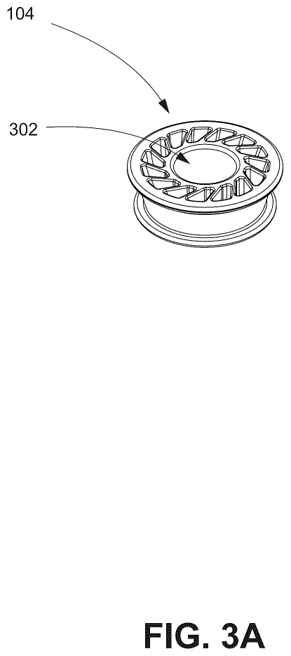

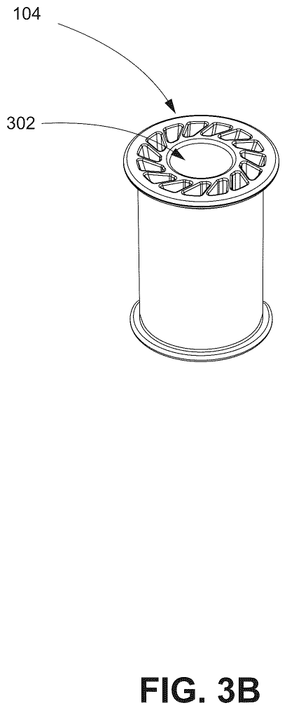

FIG. 3A is a perspective view of the flexible and bendable penile ring 104 of exemplary improved penile traction device 100 in accordance with an embodiment of the invention. The flexible and bendable penile ring 104 is generally ring-shaped, and can be made available in various configurations, but is generally designed to fit around the circumference of a human penis. The flexible and bendable penile ring 104 is also generally designed for a user to insert a penis through the cavity in the flexible ring 104. For example, in some embodiments of the invention, the flexible and bendable penile ring 104 can be made and available in various degrees of concavity, softness, diameters, and the like. In some embodiments the flexible ring may be custom molded to specifically accommodate a user's penis. In other embodiments of the invention, the flexible ring may be shaped in ways to aid in the treatment of Peyronie's disease such as, but not limited to, varying degrees of length, thickness and varying angles. In embodiments of the invention, the flexible and bendable penile ring 104 may be made from a soft material such as, but not limited to, rubber, a foam, or a silicone gel capable of softly interfacing with a human penis.

The flexible ring 104 is designed in such a way that a user may insert the glans of the penis through the center aperture 302. The user then can secure the flexible and bendable ring to the twist lock for constricting the at least one flexible and bendable penile ring 114 where the twist lock can be secured to the generally U-shaped yoke assembly 102.

FIG. 3B is a perspective view of the flexible and bendable penile ring 104 of exemplary improved penile traction device 100 in accordance with an alternative embodiment of the invention. In various embodiments of the invention, the flexible and bendable penile ring is shaped in such a way that upon constriction, a penis can be shaped in such a way to correct abnormalities. By way of example, and not limitation, a physician or practitioner can have a flexible and bendable penile ring 104 molded in such a way in which a patient suffering from Peyronie's disease can insert his penis into a specially molded flexible ring so as to correct the shape of a penis. In some embodiments the flexible ring may be custom shaped and molded to specifically accommodate varying penile lengths and widths. In other embodiments of the invention, the flexible ring may be shaped in ways to aid in the treatment of Peyronie's disease such as, but not limited to, varying degrees of length, thickness and varying angles.

In embodiments of the invention, the flexible and bendable penile ring 104 may be made from a soft material such as, but not limited to, rubber, a foam, or a silicone gel capable of softly interfacing with a human penis. In other embodiments of the invention, the flexible and bendable penile ring 104 can be made with firmer materials such as harder rubber or a firmer foam so as to allow for shaping of a penis while engaged in traction therapy. By way of example, and not limitation, a physician or urological specialist may choose a molded foam compound for use in a combination of vacuum and penile distraction therapy. In another nonlimiting example, a physician or urological specialist may create a specialized flexible and bendable ring from a firmer compound such as a rubber compound. A physician or urological specialist can choose from a number of different compounds which may prove useful in such a therapy regimen.

In alternative embodiments, the flexible and bendable penile ring 104 can assume elongated conformations so as to allow for penile shaping in conjunction with penile traction therapy. The flexible and bendable penile ring 104 is designed in such a way that a user may insert the glans of the penis through the center aperture 302. The user then can secure the flexible ring to the twist lock for constricting the at least one flexible and bendable penile ring 114 where the base unit can be secured to the generally U-shaped yoke assembly 102.

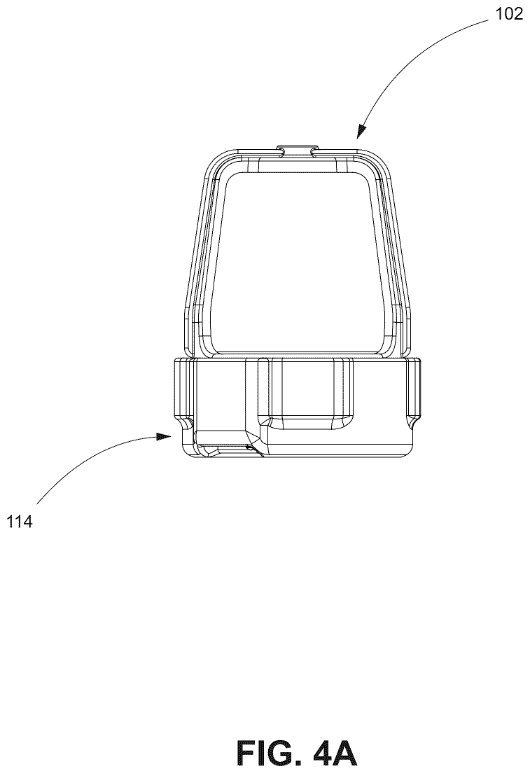

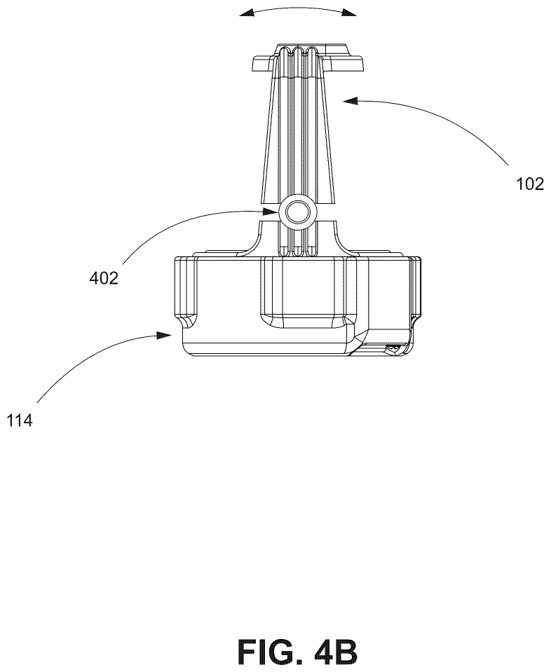

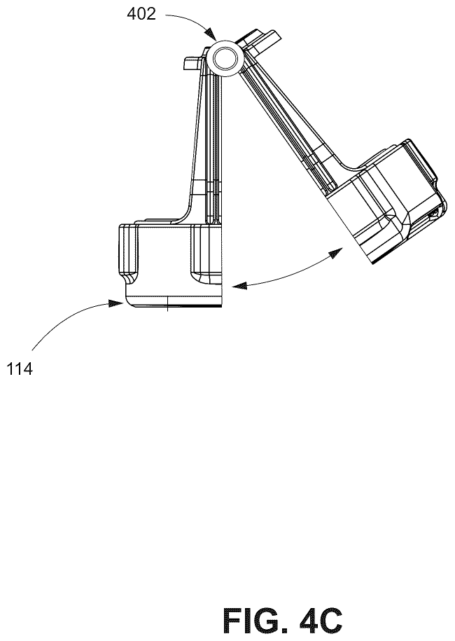

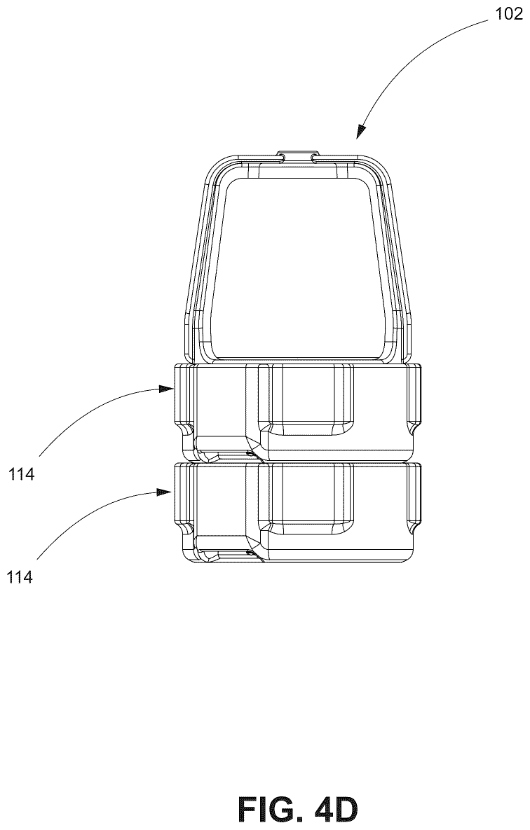

FIG. 4A is a side view of the exemplary improved penile traction device in accordance with an alternative embodiment of the invention. In embodiments of the invention, the exemplary improved penile traction device 100 comprises a generally U-shaped yoke assembly 102 having a vertex and two or more arms extending from the vertex, a flexible and bendable penile ring 104, and at least one twist lock for constricting the at least one flexible and bendable penile ring 114. In embodiments of the invention, the twist lock for constricting the flexible ring includes a plurality of cam bodies 106 and a twistable ring 108 which engages the plurality of cam bodies 106 with the flexible and bendable penile ring 104. In embodiments of the invention, the yoke assembly 102 may include two arms. However, embodiments may include four, six, eight or ten arms. In such an embodiment, the glans of a penis fits through a flexible ring where it is visible inside the space of the generally U-shaped yoke assembly 102.

FIG. 4B is a side view of the exemplary improved penile traction device in accordance with an alternative embodiment of the invention. In embodiments of the invention, the exemplary improved penile traction device 100 comprises a generally U-shaped yoke assembly 102 having a vertex and two or more arms extending from the vertex, a flexible and bendable penile ring 104, and at least one twistable ring 108 for constricting the at least one flexible and bendable penile ring, which when assembled, creates a twist lock for constricting the at least one flexible and bendable penile ring 114. In embodiments of the invention, the twist lock for constricting the flexible ring includes a plurality of cam bodies 106 and a twistable ring 108 which engages the plurality of cam bodies 106 with the flexible ring 104. In such an alternative embodiment, a hinge assembly 402 connects the generally U-shaped yoke 102 to the base unit 108 such that the at least one generally U-shaped yoke is capable of being moved laterally from one side to another of the base unit. Stated differently, the base unit includes a hinge and latch mechanism whereas the hinge and latch mechanism allows for a user to open and close the said base unit Such an alternative embodiment allows a user easier access to ensure a penis is secured inside the flexible and bendable penile ring 104 and that the twistable base unit 108 is adjusted in such a manner so as to secure the system to a penis. In such an embodiment, the glans of a penis fits through a flexible ring where it is visible inside the space of the generally U-shaped yoke assembly 102.

FIG. 4C is a side view of the exemplary improved penile traction device in accordance with an alternative embodiment of the invention. In certain alternative embodiments of the invention, the exemplary improved penile traction device 100 comprises a generally U-shaped yoke assembly 102 having a vertex and two or more arms extending from the vertex, a flexible and bendable penile ring 104, and a means for constricting the flexible ring so as to provide a secure grip on an inserted penis. In one alternative embodiment, a hinge assembly 402 connects two halves of the generally U-shaped yoke 102 and the twist lock 114. In such an alternative embodiment, a user can attach the yoke 102 and twist lock 114 to a penis by a simple clamshell mechanism. The system can be secured by a number of means such as, but not limited to, snap mechanisms, magnetic mechanisms or other mechanisms known and appreciated in the art. In such an embodiment, the glans of a penis fits through a flexible ring where it is visible inside the space of the generally U-shaped yoke assembly 102.

FIG. 4D is a side view of the exemplary improved penile traction device in accordance with an alternative embodiment of the invention. In certain alternative embodiments of the invention, the exemplary improved penile traction device 100 comprises a generally U-shaped yoke assembly 102 having a vertex and two or more arms extending from the vertex, a flexible and bendable penile ring 104, and a twist-lock means 114 for constricting the flexible ring so as to provide a secure grip on an inserted penis. In embodiments of the invention, the means for constricting the flexible ring includes a plurality of cam bodies 106 and more than one a twistable base unit 108 which engages the plurality of cam bodies 106 with the flexible and bendable penile ring 104. In such an embodiment, the glans of a penis fits through a flexible ring where it is visible inside the space of the generally U-shaped yoke assembly 102. In such alternative embodiments, the use of multiple means for constricting the flexible and bendable penile ring 104 allows a user to create shaping forces on a penis so as to correct abnormalities.

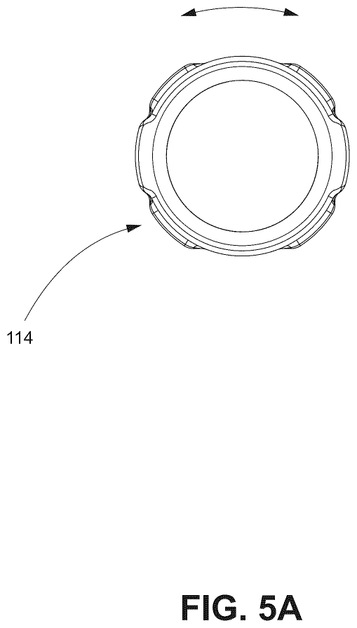

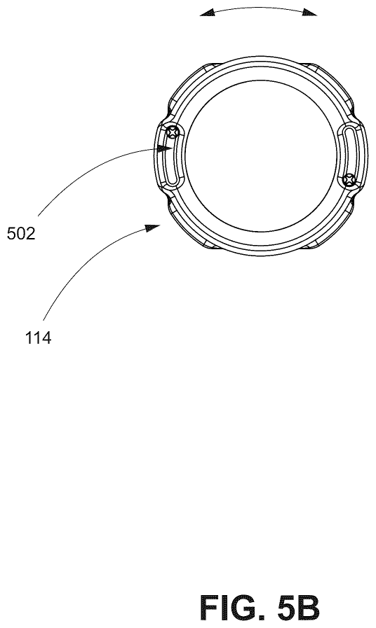

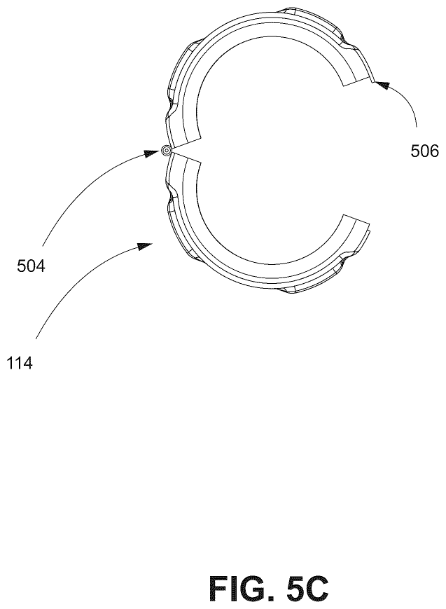

FIG. 5A is a bottom view of the base unit of the exemplary improved penile traction device in accordance with an embodiment of the invention. Persons skilled in the art will appreciate that other means of fastening the twist lock 114 to the generally U-shaped yoke assembly 102 can be used. In embodiments of the invention, the twist lock 114 may also be made from a medical-grade polycarbonate resin plastic. However, persons skilled in the art will readily appreciate that alternative embodiments may be made from a number of materials such as, but not limited to, metal and metal alloys, clear acrylic plastics, ABS plastic, polycarbonates, polyvinyl chloride (PVC), or various other plastic equivalents. Likewise, the cam bodies 106 may also be made from a medical-grade polycarbonate resin plastic or materials such as, but not limited to, metal and metal alloys, clear acrylic plastics, ABS plastic, polycarbonates, polyvinyl chloride (PVC), or various other plastic equivalents.

FIG. 5B is a bottom view of the base unit of the exemplary improved penile traction device in accordance with an alternative embodiment of the invention. A means for fastening the twist lock 114 to the generally U-shaped yoke assembly 102 can include two screws 110. Such a mechanism allows for greater strength and twistability.

FIG. 5C is a bottom view of the base unit of the exemplary improved penile traction device in accordance with an alternative embodiment of the invention. In alternative embodiments of the invention, the twist lock 108 further comprises a hinge 504 and latch 506 mechanism whereas said hinge and latch mechanism allows for a user to open and close the said base unit. Persons having skill in the art will readily appreciate that numerous hinge and latch mechanisms are available for securing a base unit together. Such a configuration allows for easier insertion of a flexible and bendable ring with a penis inserted into the ring.

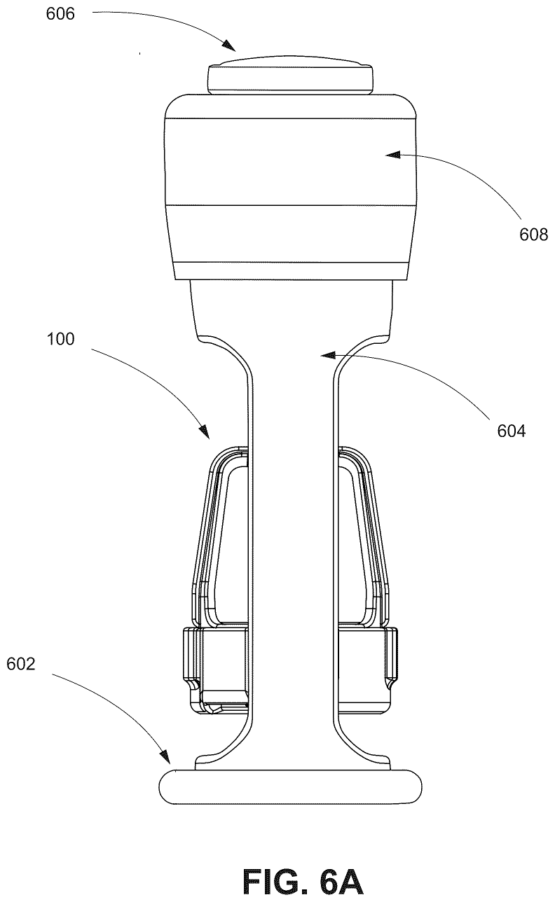

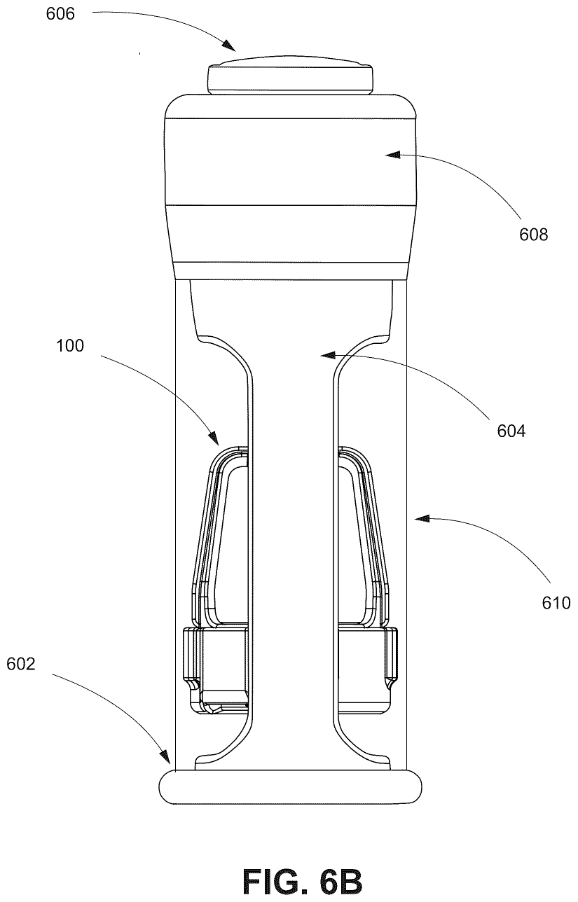

FIG. 6A is a side view of an exemplary improved penile traction device utilized in a penile traction device in accordance with an alternative embodiment of the invention. In one embodiment of the invention, the improved penile traction device is utilized in a system which comprises a base unit 602, a frame 604, a distraction means 606, and a distal end 608. In such an embodiment, an erectile dysfunction treatment system may be created and used without a vacuum cylinder for distraction therapy alone. The distal end 608 and base unit 602 attach to the frame 604. A hand dial or crank for moving the distraction means 606 and the improved penile traction device 100 is located on the top of the distal end 608. Persons skilled in the art will readily appreciate that numerous hand dials or cranks may be used to move the distraction means and improved penile traction device depending on the type of distraction means used. An attachment point 112 is located generally at the vertex of the generally U-shaped yoke assembly 102. For example, and not by way of limitation, a common star nut may attach to a bolt for a distraction means for moving the improved penile traction device 100. However, other attachment points and mechanisms can be utilized.

FIG. 6B is a side view of an exemplary improved penile traction device utilized in a penile traction device in accordance with an alternative embodiment of the invention. In embodiments of the invention, a system for penile traction therapy combined with vacuum therapy comprises a base unit 602, a frame 604, a distraction means 606, and a distal end 608. In various embodiments of the invention the cap assembly attaches to the frame. The base unit 602 also attaches to the frame 604. A vacuum cylinder unit 610 may slide over the frame 104, or can be created with two snap-on units, which rest on the base unit 602 and the cap assembly 610 then may slide over the vacuum cylinder to attach to the frame. The vacuum cylinder unit 612 is held in place by a gasket located in the cap assembly and the base unit. When a vacuum is applied to the vacuum cylinder unit 612, the exemplary erectile dysfunction treatment system is sealed in such a manner so as to maintain a vacuum for a therapeutic amount of time.

Use of the improved penile traction device is designed to be simple and minimally invasive. Use involves the user preparing the flexible and bendable penile ring 104 by cleaning and removing any foreign material. The user then prepares and inserts the penis into the flexible and bendable penile ring 104 by sliding the penis through the aperture in the flexible and bendable penile ring 104. Preparation of the penis may include, but is not limited to, cleaning and or wrapping the penis in bandages if necessary. The user then secures the penis in base assembly by engaging the plurality of cam bodies 106 through by twisting the twist lock 114 which engages the cams and constriction the flexible ring. The user may then secure the improved traction device to one of many distraction therapy devices. Such distraction devices could be simple weights attached to the improved penile traction device or the device could be attached to a mechanical distraction device which can apply a distractive force to the improved penile traction device attached to a human penis. Optionally, the user may employ a vacuum cylinder or vacuum frame seal attachments to provide contemporaneous vacuum therapy. Once the penis is secured and the, the user is then able to move improved penile traction device so as to apply a distractive force to an inserted and secured penis. The user may also apply a vacuum to the inserted and secured penis and allowing distractive force and vacuum to operate for a therapeutic amount of time. Upon completion of the therapeutic amount of time, or when the user experiences discomfort, the device may be easily detached by loosening the distraction means and/or releasing vacuum.

The exemplary improved penile traction device may be sold as a single unit or as a kit with interchangeable components. For example, differing base units with differing numbers and sizes of cams may be made for differing users with differing needs. By way of example, and not limitation, a physician or other urological specialist may prescribe differing types of flexible and bendable penile rings depending on a patient's needs. Variable courses of therapy can be prescribed as well. By way of example, a physician may advise a patient to use different types of bendable and flexible penile rings for distraction therapy alone, whereas a different type of bendable and flexible penile ring may be used for vacuum therapy combined with distraction therapy. Differing types of threads can be used to determine how far the twist lock can twist. In other embodiments, differing yoke assemblies with differing attachment point types are contemplated within the scope of the invention. In yet other embodiments, vacuum tube assemblies or frame seal attachments may be sold.

Having fully described at least one embodiment of the exemplary improved penile traction device, other equivalent or alternative methods of implementing the improved penile traction device according to the present invention will be apparent to those skilled in the art. Various aspects of the invention have been described above by way of illustration, and the specific embodiments disclosed are not intended to limit the invention to the particular forms disclosed. The particular implementation of the improved penile traction device may vary depending upon the particular context or application.

By way of example, and not limitation, the improved penile traction device described in the foregoing patent application is principally directed towards using known distraction means and methods. However, other techniques may instead be applied vacuum pump systems which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Although specific features of the invention are shown in some drawings and not others, persons skilled in the art will understand that this is for convenience. Each feature may be combined with any or all of the other features in accordance with the invention. The words “including,” “comprising,” “having,” and “with” as used herein are to be interpreted broadly and comprehensively, and are not limited to any physical interconnection. Claim elements and steps herein may have been numbered and/or lettered solely as an aid in readability and understanding. Any such numbering and lettering in itself is not intended to and should not be taken to indicate the ordering of elements and/or steps in the claims to be added at a later date.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Any amendment presented during the prosecution of the application for this patent is not a disclaimer of any claim element presented in the description or claims to be filed. Persons skilled in the art cannot reasonably be expected to draft a claim that would literally encompass each and every equivalent.

What is claimed is:

1. An improved penile traction device comprising:
At least one generally U-shaped yoke, each U-shaped yoke having a vertex and two arms;
at least one flexible and bendable penile ring capable of interfacing with a human penis; and
at least one twist lock for constricting the at least one flexible and bendable penile ring, said twist lock including a base unit having a plurality of cam bodies and a twistable ring which engages the plurality of cam bodies.

2. The improved penile traction device of claim 1 further comprising an attachment point located at the vertex of the at least one generally U-Shaped yoke assembly, said attachment point capable of interfacing with a penile distraction device.

3. The improved penile traction device of claim 1 wherein the at least one twist lock for constricting the at least one flexible and bendable penile ring is adjustable so as to provide sufficient gripping force on a penis so as to remain engaged with a human penis when a distractive force is applied.

4. The improved penile traction device of claim 1 wherein the at least one flexible and bendable penile ring is shaped in such a way that upon constriction, a penis can be shaped.

5. The improved penile traction device of claim 1 wherein the at least one flexible and bendable penile ring is made from a rubber compound.

6. The improved penile traction device of claim 1 wherein the at least one flexible and bendable penile ring is made from a foam compound.

7. An improved penile traction device for a penile traction device, said device configurable for a combination of vacuum therapy and traction therapy, said device comprising:
At least one generally U-shaped yoke, each U-shaped yoke having a vertex and two arms;
at least one flexible and bendable penile ring capable of interfacing with a human penis; and
at least one twist lock for constricting the at least one flexible and bendable penile ring, said twist lock including a base unit having a plurality of cam bodies and a twistable ring which engages the plurality of cam bodies.

8. The improved penile traction device of claim 7 further comprising an attachment point located at the vertex of the said yoke assembly, said attachment point capable of interfacing with the distraction means of a penile distraction device.

9. The improved penile traction device of claim 7 wherein the improved yoke assembly is capable of fitting inside a cylindrically shaped vacuum distraction therapy device.

10. The improved penile traction device of claim 7 wherein the at least one twist lock for constricting the at least one flexible and bendable penile ring is adjustable so as to provide sufficient gripping force on a penis so as to remain engaged with a human penis when a distractive force is applied.

11. The improved penile traction device of claim 7 wherein the at least one flexible and bendable penile ring is shaped in such a way that upon constriction, a penis can be shaped.

12. The improved penile traction device of claim 7 wherein the at least one flexible and bendable penile ring is capable of expanding so as to apply a shaping force on a human penis when a partial vacuum is applied.

13. The improved penile traction device of claim 7 wherein the at least one flexible and bendable penile ring is made from a rubber compound.

14. The improved penile traction device of claim 7 wherein the at least one flexible and bendable penile ring is made from a foam compound.

15. An improved penile traction device useful for penile distraction devices, said device comprising:
At least one generally U-shaped yoke, each U-shaped yoke having a vertex and two arms;
at least one flexible and bendable penile ring capable of interfacing with a human penis; and
at least one adjustable twist lock for constricting the at least one flexible and bendable penile ring, said twist lock including a base unit having a plurality of cam bodies and a twistable ring which engages the plurality of cam bodies.

16. The improved penile traction device of claim 15 further comprising an attachment point located at the vertex of the said yoke assembly, said attachment point capable of interfacing with one or more weights, said weights used to apply a distractive force on a human penis.

17. The improved penile traction device of claim 15 wherein the at least one twist lock for constricting the at least one flexible and bendable penile ring is adjustable so as to provide sufficient gripping force on a penis so as to remain engaged with a human penis when a distractive force is applied.

18. The improved penile traction device of claim 15 wherein the at least one flexible and bendable penile ring is shaped in such a way that upon constriction, a penis can be shaped in such a way to correct abnormalities.

19. The improved penile traction device of claim 15 wherein the at least one generally U-shaped yoke further comprises a hinge assembly which connects the generally U-shaped yoke to the base unit such that the at least one generally U-shaped yoke is capable of being moved laterally from one side to another of the base unit.

20. The improved penile traction device of claim 15 wherein the base unit further comprises a hinge and latch mechanism whereas said hinge and latch mechanism allows for a user to open and close the said base unit.
